(19) United States
(12) Patent Application Publication
(10) Pub. No.: US 2022/0175307 A1
(43) Pub. Date: Jun. 9, 2022

(54) DRESSINGS, SYSTEMS AND METHODS FOR PHLEBITIS DETECTION

(71) Applicant: Smiths Medical ASD, Inc., Plymouth, MN (US)

(72) Inventors: David J. Goral, Plymouth, MN (US); James M. Muskatello, Plymouth, MN (US)

(73) Assignee: Smiths Medical ASD, Inc., Plymouth, MN (US)

(21) Appl. No.: 17/600,047

(22) PCT Filed: May 21, 2020
(86) PCT No.: PCT/US2020/033958

§ 371 Date: Sep. 29, 2021

Related U.S. Application Data
(60) Provisional application No. 62/852,842, filed on May 24, 2019.

(51) Int. Cl. A61B 5/00; A61B 5/103; A61F 13/00; A61F 13/02

(52) U.S. Cl. CPC A61B 5/445; A61B 5/1032; A61B 5/6802; A61F 2013/0094; A61F 13/02; A61B 5/0075; A61F 13/00051

(57) ABSTRACT

A medical dressing comprises at least one light source and at least one light sensor fixedly attached to a substrate and configured to measure a reddening of a portion of skin as an early indicator of phlebitis around the wound site. A method of phlebitis detection and monitoring comprises covering a wound site with a medical dressing comprising at least one light source and at least one light sensor such that the wound site is situated between the two, and whereby reddening of the skin is monitored by analyzing light reflected off the skin.

### CROSS-REFERENCE TO RELATED APPLICATIONS

This application claims priority to and the benefit of U.S. Provisional Patent Application Ser. No. 62/852,842 filed May 24, 2019 and entitled “Dressings, Systems and Methods for Phlebitis Detection,” the disclosure of which is incorporated herein by reference in its entirety for all purposes.

### FIELD

The present disclosure relates generally to medical devices, and more specifically, to a device comprising sensors configured to detect and monitor a medical condition.

### BACKGROUND

Phlebitis is one of the highest causes for premature termination of IV infusion. The act of starting an IV triggers defense mechanisms in the patient that may sometimes result in phlebitis. Detection today is limited to periodic visual inspection of the infusion site by a clinician, or the complaint of pain from the patient. However, the site may also be partially obstructed by the securement placed over the inserted IV. Therefore, there is an ongoing need in the medical profession for new continuous monitoring systems capable of early detection and tracking the rate of phlebitis.

### SUMMARY

In various embodiments, a dressing may be configured with at least one sensor capable of detecting and monitoring phlebitis at a wound site. The sensor may be configured to measure and record changes in skin color at the wound site that relates to the extent of phlebitis or other inflammation. In various embodiments, a dressing may be configured to monitor phlebitis in peripheral vein access sites, e.g., catheter placement sites.

In various embodiments, a dressing herein may be configured to first signal a color change of the skin at the wound site. Skin color change may be the first sign a clinician would obtain by periodic visual inspections of the wound site, other than if the patient complains of localized pain at the site. An advantage of the present dressings is that a continuous monitoring system comprising the dressing will likely signal a clinician of a problem existing at the wound site earlier than the clinician might have detected through periodic observation. With constant monitoring of skin color, a shift in the health of the wound site can potentially be detected in response to the shift not yet being visible to the clinician. Further, with more than one sensor present in the dressing, the rate of phlebitis expansion can be measured. For example, data obtained by the dressing may indicate rate of skin color change at one sensor as well as the rate of skin color change from one sensor to the next, as a way to detect and estimate phlebitis expansion.

In various embodiments of the present disclosure, a dressing comprises: a substrate configured for placement over a region of skin encompassing a wound site on a patient; a first light source fixedly attached to the substrate; and a first light sensor fixedly attached to the substrate, the first light sensor spaced apart from the first light source by a distance d1; wherein the first light sensor is configured to receive light from the first light source reflected off the region of skin in response to the wound site being disposed between the first light source and the first light sensor.

In various embodiments, the first light source and the first light sensor cooperate as a reflectance spectrophotometer configured to measure a color of the region of skin. In various embodiments, the first light source may comprise a white LED or a full-color RGB LED lamp. In various embodiments, the first light sensor may comprise an RGB color sensor.

In various embodiments, the substrate comprises an adhesive portion configured to adhere the dressing to the patient.

In various embodiments, the substrate may further comprise a transparent region dimensionally sized to incorporate the first light source, the first light sensor, and the region of skin within its dimensions.

In various embodiments, the distance d1 may be from about 1 mm to about 100 mm.

In various embodiments, the first light source and the first light sensor are integrated on a circuit board having a front face whereupon the first light source and the first light sensor are exposed and a back face that is fixedly attached to the substrate.

In various embodiments, the dressing may further comprise a wiring harness configured to supply power to, and data communication with, the first light source and the first light sensor, the wiring harness further including a quick-disconnect connector along the wiring harness, adjacent the substrate.

In various embodiments, the dressing may further comprise a second light sensor fixedly attached to the substrate and spaced apart from the first light source by a distance d2, the second light sensor positioned on the opposite side of the first light source from the first light sensor such that the second light sensor, the first light source, and the first light sensor are linearly aligned, wherein the second light sensor is configured to receive light from the first light source reflected off the region of skin in response to the wound site being disposed between the first light source and the first light sensor and not being disposed between the first light source and the second light sensor.

In various embodiments, the second light sensor is configured to measure a baseline skin color of the region of skin, and wherein the first light sensor is configured to measure a change in skin color relative to the baseline skin color. In various embodiments, the combination of the first light source and the second light sensor may also be configured as a reflectance spectrophotometer.

In various embodiments, the distance d2 may be equal in length to the distance d1.

In various embodiments, each distance d1 and d2 is independently from about 1 mm to about 100 mm.

In various embodiments, the dressing may further comprise a second light source fixedly attached to the substrate and a second light sensor fixedly attached to the substrate, the second light sensor separated from the second light source by a distance d3, wherein the second light sensor is configured to receive light from the second light source reflected off the region of skin in response to the wound site being disposed between the first light source and the first light sensor and not being disposed between the second light source and the second light sensor.

In various embodiments, the first light source and the first light sensor, and the second light source and the second light sensor, are configured as independent first and second reflectance spectrophotometers, respectively.

In various embodiments, the distance d3 is equal in length to the distance d1, wherein the second light source and the first light source are separated by a distance d4, and the second light sensor and the first light sensor are separated by the same distance d4, such that the first and second light sources and the first and second light sensors are disposed in a square or a rectangular array.

In various embodiments, the distance d4 may be from about 1-200 mm.

In various embodiments, the second reflectance spectrophotometer is configured to measure a baseline skin color of the region of skin, and wherein the first reflectance spectrophotometer is configured to measure a change in skin color relative to the baseline skin color.

In various embodiments, a system for phlebitis detection and monitoring comprises: a dressing comprising: a substrate configured for placement over a region of skin encompassing a wound site on a patient; a first light source; a first light sensor; and a second light sensor, each fixedly attached to the substrate; the first light sensor spaced apart from the first light source by a distance d1; the second light sensor spaced apart from the first light source by a distance d2; and the second light sensor positioned on the opposite side of the first light source from the first light sensor such that the second light sensor, the first light source, and the first light sensor are linearly aligned; and a signal processing device in electronic data communication with each of the first light source, the first light sensor, and the second light sensor; wherein the first light sensor is configured to receive light from the first light source reflected off the region of skin in response to the wound site being disposed between the first light source and the first light sensor, and the second light sensor is configured to receive light from the first light source reflected off the region of skin in response to the wound site being disposed between the first light source and the first light sensor and not being disposed between the first light source and the second light sensor; wherein the second light sensor is configured to measure a baseline skin color of the region of skin, and wherein the first light sensor is configured to measure a change in skin color relative to the baseline skin color; wherein the signal processing device provides an output in response to a change from the baseline skin color; and wherein the output comprises at least one of a visual indicator, an audible indicator, a message sent, and an automated change to the conditions at the wound site.

In various embodiments of the system, the first light source, the first light sensor and the second light sensor are integrated on a circuit board, the circuit board electrically powered by a power supply disposed in the signal processing device.

In various embodiments, a method of detecting and monitoring the rate of phlebitis at a region of skin encompassing a wound site on a patient comprises: covering the region of skin with a dressing comprising a first light source and a first light sensor spaced apart from the first light source by a distance d1, such that the wound site is disposed between the first light source and the first light sensor; and obtaining a color of a portion of skin in the region of skin between the first light source and the first light sensor by measuring light from the first light source reflected off the portion of skin over time; wherein the dressing further comprises a substrate dimensionally configured to cover the region of skin and onto which the first light source and the first light sensor are fixedly attached; wherein a red color thus obtained indicates the presence of phlebitis; and wherein a reddening of skin color over time indicates a worsening of the phlebitis.

In various embodiments of the method, the first light source and the first light sensor cooperate as a reflectance spectrophotometer configured to measure the color of the portion of skin in the region of skin between the first light source and the first light sensor.

In various embodiments of the method, the first light source comprises a white LED or a full-color RGB LED lamp.

In various embodiments of the method, the dressing further comprises a second light sensor fixedly attached to the substrate and spaced apart from the first light source by a distance d2, the second light sensor positioned on the opposite side of the first light source from the first light sensor such that the second light sensor, the first light source, and the first light sensor are linearly aligned, wherein the second light sensor is configured to receive light from the first light source reflected off the region of skin in response to the wound site being disposed between the first light source and the first light sensor and not being disposed between the first light source and the second light sensor.

In various embodiments, the method further comprises measuring a baseline skin color of a portion of skin in the region of skin between the first light source and the second light sensor, and measuring a color of the portion of skin in the region of skin between the first light source and the first light sensor over time relative to the baseline skin color.

In various embodiments of the method, the distance d1 may be equal to the distance d2.

In various embodiments of the method, the dressing further comprises a second light source fixedly attached to the substrate and a second light sensor fixedly attached to the substrate, the second light sensor separated from the second light source by a distance d3, wherein the second light sensor is configured to receive light from the second light source reflected off the region of skin in response to the wound site being disposed between the first light source and the first light sensor and not being disposed between the second light source and the second light sensor.

In various embodiments of the method, the first light source and the first light sensor, and the second light source and the second light sensor, are configured as independent first and second reflectance spectrophotometers, respectively.

In various embodiments of the method, the distance d3 is equal in length to the distance d1, and wherein the second light source and the first light source are separated by a distance d4, and the second light sensor and the first light sensor are separated by the same distance d4, such that the first and second light sources and the first and second light sensors are disposed in a square or a rectangular array.

In various embodiments, the method may further comprise measuring a baseline skin color of a portion of skin in the region of skin between the second light source and the second light sensor, and measuring a color of the portion of skin in the region of skin between the first light source and the first light sensor over time relative to the baseline skin color.

### BRIEF DESCRIPTION OF THE FIGURES

The subject matter is pointed out with particularity and claimed distinctly in the concluding portion of the specification. A more complete understanding, however, may best be obtained by referring to the detailed description and claims when considered in connection with the following drawing figures:

FIG. 1A illustrates examples of a dressing for phlebitis detection and phlebitis monitoring comprising an arrangement of one light source and two sensors, in accordance with various embodiments;

FIG. 1B illustrates a magnified view of an integrated circuit board portion of the dressing in FIG. 1A, comprising a spaced apart arrangement of one light source and two sensors, wherein the dressing is located over a wound such that the wound is between the light source and one of the sensors, in accordance with various embodiments;

FIG. 1C illustrates examples of a dressing for phlebitis detection and monitoring comprising an arrangement of two light sources and two sensors arranged in pairs such that one light source/sensor pair functions as a reference, in accordance with various embodiments;

FIG. 2 illustrates examples of a system for phlebitis detection and monitoring comprising a dressing with sensors electronically connected to a controller, in accordance with various embodiments;

FIGS. 3A-3C progressively illustrate various examples of a method for phlebitis detection and monitoring using the arrangement of one light source and two sensors as per FIG. 1A/1B, in accordance with various embodiments; and

FIGS. 4A-4C progressively illustrate various examples of a method for phlebitis detection and monitoring using the arrangement of one light source and two sensors as per FIG. 1C, in accordance with various embodiments.

### DETAILED DESCRIPTION

The detailed description of various embodiments herein references the accompanying drawings, which show various embodiments by way of illustration. While these various embodiments are described in sufficient detail to enable those skilled in the art to practice the disclosure, it should be understood that other embodiments may be realized, and that logical, chemical, mechanical and structural changes may be made without departing from the spirit and scope of the disclosure. Thus, the detailed description herein is presented for purposes of illustration only and not of limitation.

In various embodiments, a dressing for detecting the presence of phlebitis at a wound site of a patient is described. In various embodiments, the dressing is configured to detect and/or monitor the extent and/or the rate of progression of phlebitis at a wound site of a patient. In various embodiments, a dressing configured to detect and/or monitor the extent of phlebitis over time comprises at least one light source and a light sensor (i.e., an optical receiver) configured to measure skin color and/or to monitor changing skin color over time. Although embodiments herein describe dressings and methods for detecting and monitoring phlebitis by skin color and skin color changes over time, it should be understood that similar dressings are included within the scope of the present disclosure that can detect and/or monitor the extent and/or the rate of progression of phlebitis based on temperature and/or temperature changes over time of a wound site of a patient, rather than skin color and/or skin color changes over time at the wound site by replacement of color sensors with temperature sensors. For example, a dressing for detecting and/or monitoring phlebitis may comprise temperature sensors that are placed in contact with or in close proximity to the skin in response to the dressing being applied to the wound site to be monitored.

In various embodiments, a dressing usable to detect and/or monitor the extent and/or the rate of progression of phlebitis at a wound site of a patient comprises a substrate, (e.g., in the form of an adhesive patch), at least one light source fixed to the substrate, and at least one light sensor fixed to the substrate. In various embodiments, the dressing functions as both a wound covering and a miniaturized reflectance spectrophotometer configured to provide an assessment of the color of a region of skin between a light source and a light sensor over time. In various embodiments, light emitted from the light source reflects off a region of skin at and around the wound site and is received in the light sensor for analysis. Color is determined by light reflection off the skin surface, rather than light transmission through skin or tissue.

### Definitions and Conventions

As used herein, the term “wound site” refers broadly to a skin surface at a surgical wound site (e.g., a catheter tube insertion site, a medical device port site, a vaccination site, an incision site, etc.), a non-surgical wound site (e.g., a cut, abrasion, hematoma), or any other site on or below a skin surface to be monitored for phlebitis. In this regard, a wound site can be a venous access point, such as for example, an injection site into a basilic or cephalic vein. Exemplary embodiments and associated drawing figures herein may focus on a catheter tube peripheral vein insertion site as the wound site to monitor for phlebitis, but the scope of the present disclosure should not be interpreted as being limited for use in monitoring only this particular surgical wound site. In various drawings, a wound site might be illustrated as a generic cut, but it should be understood the drawings are not so limited, and that a wound may be an incision through which a catheter tube is still placed. In those applications, the dressings of the present disclosure may be simply rotated as needed before placing on the patient such that one of the light sources or light sensors is not directly on top of a catheter tube emerging from the wound site.

As used herein, the term “phlebitis” takes on a broader meaning that its ordinary meaning in the medical profession, so as to include inflammation in general. The symptoms of phlebitis generally include redness, warmth and/or pain in the affected area, i.e., the wound site, and it is one or more of these symptoms that are detected and monitored by the dressing and methods disclosed herein. Additionally, while the present disclosure is described with reference to detecting and monitoring phlebitis, which is venous inflammation, other similar conditions may be monitored using the dressings, systems and methods herein. For example, inflammation and infection at any surgical or non-surgical wound site can be monitored by the dressings herein. For example, infection at a site of a surgical incision or a repair site of trauma may be monitored for phlebitis with the dressings, systems and methods disclosed herein.

As used herein, the term “dressing” refers to a medical device configured to detect and/or monitor the rate of phlebitis at a wound site. The term is used to indicate the medical device has the general appearance of a medical dressing, like a patch. The term “dressing” is used for the devices herein since, in various embodiments, a dressing capable of detecting and/or monitoring the rate of phlebitis at a wound site in a patient is in the physical form of a covering for the wound site (i.e., a dressing—like a patch or a bandage). As discussed in more detail herein, a dressing in accordance with the present disclosure is a medical device usable as a covering for a wound site, further comprising sensors configured to detect and monitor phlebitis. Therefore, a “dressing” herein comprises a substrate further comprising a patch, a gauze, a tape, a bandage, a covering, a pad, or a monitor or any other physically stabilizing platform for fixedly positioning at least one sensor configured to detect and monitor phlebitis, as described herein.

General Embodiments of a Dressing for Phlebitis Detection and/or Monitoring

In various embodiments, a dressing herein for phlebitis detection and monitoring comprises a substrate, for example in the form of an adhesive patch or bandage, and at least one sensor fixed to the substrate. In various embodiments, a wiring harness is connected to the at least one sensor, and the harness may enter the substrate at one point or simply lay underneath and connect to the at least one sensor. As explained in more detail herein, the wiring harness may be detachable from the dressing (e.g., through a quick-disconnect connector) so that part of the dressing can be disposed of after use, while other portions can be kept and reused. For example, the entire substrate along with the at least one sensor attached to the substrate, and one end of the wiring harness, e.g., a portion from the quick-disconnect connector to the sensor, may be disposable. After use, a clinician would remove the dressing from the wound site, disconnect the wiring harness from the dressing by way of the quick-disconnect connector, and dispose of the dressing. In various embodiments, the quick-disconnect connector is provided along the wiring harness adjacent to the substrate of the dressing such that only a small portion of the wiring harness leading into the sensors is discarded with the dressing. An exemplary quick-disconnect connector may be part of a set of patch cables that include quick-disconnect plugs, such as M12 axial male and female having 5 or more poles, depending on the desired number of data communication connections along with the power and ground. There are countless options for wiring harnesses and quick-disconnect connectors for use in the dressings of the present disclosure.

In various embodiments, at least one sensor is integrated into a circuit board (e.g., a small board with electronic components wired thereon, wherein the board may be less than about 20 mm×20 mm and less than about 2 mm thick). In various embodiments, a circuit board configuration allows for at least one light source and at least one sensor to be arranged and positionally fixed at particular distances from each other, and allows a simple electronic connection by way of a wiring harness from a power supply and optional data recording module to the pin connections provided on the integrated circuit board. In various embodiments including a circuit board format, it is the circuit board that is fixedly attached to the substrate of the dressing rather than individual lights and sensors. In response to a circuit board being utilized, it should be understood that an observer might not be able to see the various light sources and light sensors in response to a dressing being placed on a patient, because in various embodiments, the circuit board might be inverted over the wound site such that the electronic components on top of the circuit board are placed adjacent the skin and ultimately not visible. Various drawings herein are simplified for clarity purposes by elimination of the wiring details to the individual light and sensor components along with disregard for instances where the wound and the electronic components may all be hidden on the other side of an integrated circuit board. Various drawings herein focus on the relative positioning of the light sources, light sensors and the wound site, and how expanding inflammation from the wound site may encroach into zones between light source and light sensor. Therefore, in various drawings provided herein, the option of a circuit board format for integrating lights and sensors is represented only as a square or rectangular dashed outline so as not to over complicate the figures.

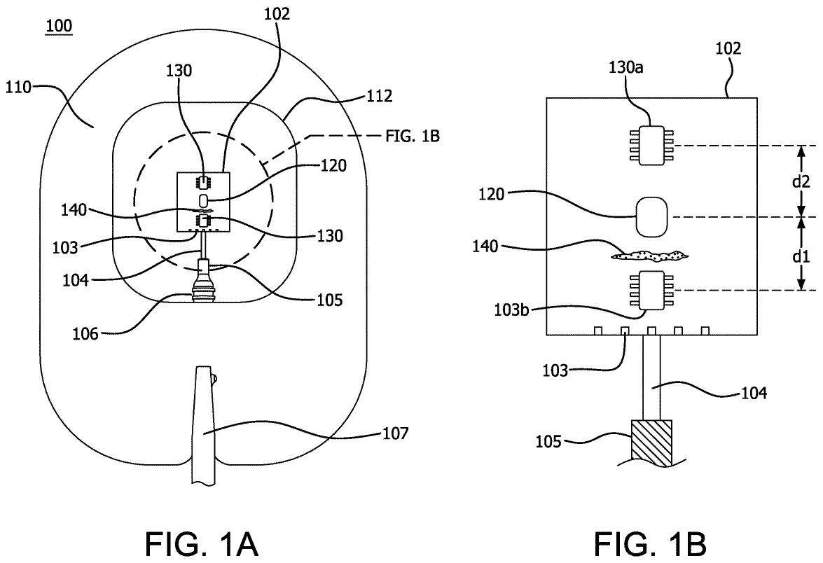

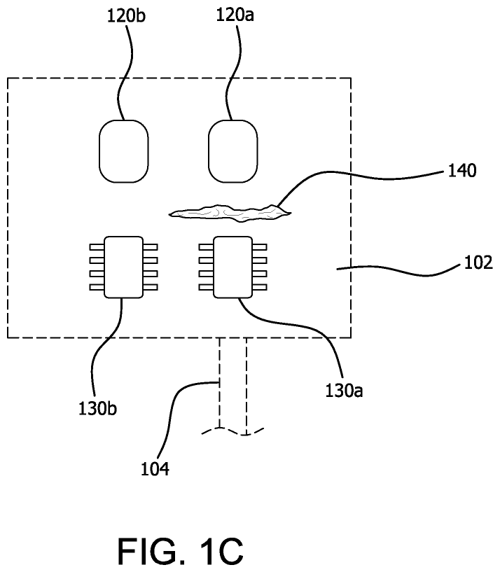

With reference now to FIG. 1A, a dressing 100 for phlebitis detection comprises a substrate 110, at least one light source 120 fixed to the substrate, and at least one sensor 130 fixed to the substrate. In this illustrated example, the wound site 140 is shown as a small surgical wound site, such as a 1-5 mm long×1 mm wide incision made laterally on an arm of a patient, such as might be the result of placement of a catheter or the stitching of a laceration. This illustration is not meant to be limiting, as the wound site 140 may be non-surgical, like a contusion having more of a circular shape. In the illustrated example of FIG. 1A, the dressing 100 comprises one light source 120 and two sensors 130, the two sensors 130 arranged on opposite sides of the light source 120 such that the three electronic components are linearly aligned. As discussed in more detail herein below, a relatively close proximity between the light source 120 and each of the associated sensors 130, and the linear arrangement of the three components, can be the result of integrating the three components in an electronic package, i.e., a circuit board.

In FIG. 1A, the one light source 120 and two sensors 130 are shown integrated on a circuit board 102, the sensors shown with connection pins to the circuit board. As mentioned, the overall circuit board 102 may be fixedly attached to the substrate 110 rather than the individual light source and sensors being fixed to the substrate 110. Further, the backside of the circuit board 102 may be fixed to the substrate so that the electronic components can be brought close to the skin in response to the substrate being placed on the patient. Not illustrated are optional ridges configured on the circuit board 102 on the same side as the light source and sensors to elevate the light source and sensors from the skin in response to the circuit board being inverted and placed against the patient. These ridges might be about 0.06 inch (about 1.5 mm) to about 0.10 inch (about 2.5 mm) in height off the surface of the circuit board. The circuit board 102 is shown with pin connectors 103 along one edge, where a short electrical lead 104 is provided between the circuit board 102 and a first portion 105 of an electrical coupling 106. The other end of the electrical coupling 106 is the remainder of the wiring harness 107 for the dressing, discussed in more detail herein below. In FIG. 1A, the wound site 140 is approximately centered between the light source 120 and one of the sensors 130. The illustrated electronic details are not meant to be limiting in any way.

In various embodiments, the substrate 110 is configured to cover a wound site, such as hygienically with a sterile barrier. In various embodiments, the substrate 110 may also advantageously hold a catheter or other inserted medical device like a Foley catheter in place and against the patient. As described in more detail herein, the substrate 110 may further comprise an opening or transparent section 112, such as positioned toward the middle of the dressing as illustrated, so that the wound site remains visible even though the wound site is covered by the dressing. In instances where a circuit board format might obscure the wound site, the circuit board 102 may be configured with an opening, i.e., not a contiguous flat square or rectangular board, or may be made at least partly of a transparent material. In various embodiments, the substrate 110 provides a medical covering for any surgical or non-surgical wound site, regardless of whether a medical device, such as a catheter, remains at the wound site and is also secured by the substrate 110. In other words, the dressing 100 provides both a substrate 110 for hygienically covering a wound site and the necessary electronic components to detect and monitor inflammatory changes to the wound site.

The substrate 110 is generally configured to facilitate placement of the dressing 100 in close proximity to a wound site 140, and in some instances to hygienically cover the wound site. In this regard, the substrate 110 can comprise an adhesive on one side of a patch shape configured to secure the substrate 110 over a wound site 140. In various embodiments, the substrate 110 may comprise a sterile dressing with an adhesive around the periphery. In various embodiments, the substrate 110 comprises a doughnut shaped opaque adhesive portion surrounding a transparent non-adhesive portion. In various embodiments, the non-adhesive portion may be transparent, such as comprising two layers of transparent tape or film in between which the light source(s) sensor(s) can be securely fixed, or between which an integrated circuit board can be fixed into position.

In various embodiments, a portion of the substrate 110 comprises markings to facilitate alignment of the substrate with an underlying wound site so that the light source and sensors (e.g., 120 and both of 130), fixedly positioned on, or within, the substrate 110, end up correctly positioned at the wound site 140 in response to the substrate being applied to the wound site 140. In instances where the light source and sensors are packaged on a circuit board, the alignment markings may be configured on the back of the circuit board, such as on opposite edges, so that a clinician can align the markings with the underlying wound 140 that might not remain visible as the dressing is applied to the patient. In the configuration illustrated in FIG. 1A, the dressing 100 is applied to the wound site 140 such that the wound site 140 is approximately evenly spaced between the light source 120 and one of the light sensors 130. In this way, the light reflected across the wound site 140 to the adjacent sensor can be compared to light reflected to the other sensor that is not positioned across the wound site but is instead positioned on the other side of the light source 120.

With continued reference to FIG. 1A, in various embodiments, the light source 120 and the light sensors 130 may be physically attached and positionally fixed to the substrate 110, for example, within the non-adhesive and transparent portion, remaining visible therethrough in response to the dressing being applied to the wound site. In various embodiments, the light source 120 and the light sensors 130 may be integrally packaged as a single electronic device like a circuit board, and that circuit board is fixed to the substrate in a particular location. In use, with the dressing applied to the wound site by virtue of the substrate, the light source 120 and the light sensors 130 are ideally positioned about 0.06 inch (about 1.5 mm) to about 0.10 inch (about 2.5 mm) off the surface of the skin. If the light source and associated sensor(s) are too close to the skin, e.g., closer than about 0.06 inch (1.5 mm), the skin can block the light from the light source, and also block the light reflected to the one or more sensors. In other words, if these components are pressed into the skin, the skin acts as a barrier between the light source and light sensor rather than a surface from which the light can reflect. If the light source and associated sensor(s) are too far away from the skin, e.g., further away than about 0.10 inch (2.5 mm), the reflected light signal may become too attenuated.

FIG. 1B illustrates a magnified view of the sensor portion (indicated in FIG. 1A as a dashed circle) of a dressing in accordance with various embodiments. In this illustration, a circuit board 102, comprising one light source 120 and two sensors 130a and 130b, is powered and communicated with by electronic leads 104 connected to the pins 103 of the circuit board, with the electronic leads 104 connected to one portion 105 of a quick-disconnect connector. The circuit board 102 packaging of the components is optional, as the electronic components can be fixedly attached, individually, to the substrate, and individual wired as necessary with both power and data communication connections. However, use of a circuit board allows the positions of the components to be fixed prior to the circuit board being fixedly attached to the substrate portion of the dressing. That is, the distances d1 and d2 between the components can be permanently set by virtue of packaging the components on a circuit board 102. The two sensors 130a and 130b are illustrated with 8-pin connectors, but this is not meant to be limiting, recognizing the number of pins may vary for different light sensors. The light source 120 may be a LED or other light source, and may be square, round or another shape other than what is illustrated. In various embodiments, the electronic components are placed in close proximity to the wound site 140 such that the wound site 140 is about centered between the light source 120 and one light sensor 130b. Certainly, there are instances where the wound cannot be precisely centered because of an irregular shape or size to the wound, or if a circuit board is configured with fixed distances d1 and d2 that happen to be too short for the task at hand.

In various embodiments, for a wound site 140 comprising about a 1-5 mm long×1 mm wide incision, d1 and d2 may independently be from about 1-100 mm. For various surgical and non-surgical wound sites, the light source 120 and sensors 130a and 130b can be brought closer together (e.g., <1 mm) or spaced further apart (e.g., >100 mm). The range of spacing may change depending on base skin color of the patient (e.g., racial variation), skin texture, the incident angle of the light source, and attenuation. In various embodiments, a clinician may have a repertoire of dressings with fixedly attached circuit boards having various d1,d2 spacing, and the clinician picks the appropriate dressing for the wound. In various embodiments, the wound site 140 may occupy a substantial portion of, or even the entirety of the space between a light source and a light sensor.

In various embodiments, the light source 120 provides the lighting for the sensor 130 to accurately measure skin color at the wound site 140. In various embodiments, lighting from the light source 120 originates at or adjacent to the wound site 140. In other embodiments, light may originate at a location spaced apart from the wound site 140, whereupon it is transferred to the wound site, for example, along an optical fiber.

In various embodiments, light source 120 is configured to provide light anywhere on the electromagnetic spectrum, including visible light (i.e., a plurality of different and distinct wavelengths within the visible light spectrum). Example light sources for use in accordance with the present disclosure include, but are not limited to, light emitting diodes (LEDs), white light, lasers, ultraviolet sources and infrared sources. In various embodiments, the light source 120 comprises an LED, such as a white LED or a full-color RGB (red, green blue) LED lamp. Various LEDs may also be chosen in regard to the luminous intensity (1v) and/or the viewing angle of the LED. The viewing angle can be leveraged so that light received by the light sensor must have been reflected off the skin rather than simply transmitted across and parallel the skin surface without first reflecting off the skin. In instances where the light source(s) and light sensor(s) are integrated on a circuit board, the light sensor(s) can be elevated relative to the light source(s) such that direct incident light from a light source to a light sensor is not possible because the light would be incident on the side of the light sensor “chip.”

In various embodiments, the light source comprises a white LED measuring about 0.063 inch (1.6 mm)×0.063 inch (1.6 mm) square. In various embodiments, the white LED source may be round with a domed top rather than square. These miniature LED light sources for use herein may be obtained, for example, from Digi-Key Electronics, Thief River Falls, Minn., USA. These components are generally powered at about 1-6V, and generally from 2.7-5.5V. The power is supplied via the wiring harness 107 discussed herein.

In various embodiments, the sensor 130 is fixedly attached to the substrate 110, for example, at the non-adhesive portion, and visible therethrough. In general, the sensor 130 measures skin color (e.g., redness) at the wound site 140. In various embodiments, the sensor 130 measures color at the dermis and/or epidermis. In various embodiments, the sensor 130 does not measure color within or below subcutaneous tissue. In accordance with various embodiments of the present disclosure, the sensor 130 measures skin color independent of the presence or absence of IV infiltration or extravasation.

In various embodiments, the sensor 130 measures skin color at predefined intervals or continuously. The sensor 130 may measure light anywhere on the electromagnetic spectrum, including exclusively visible light (i.e., a plurality of different and distinct wavelengths within the visible spectrum). Example sensors for use in accordance with the present disclosure include those that detect light emitted from LEDs, white light sources, lasers, ultraviolet sources and infrared sources. In various embodiments, a white LED source is used in conjunction with a three light intensity sensor with a color filter (RGB sensor).

In various embodiments, the sensor 130 comprises a color light sensor. In various embodiments, the sensor 130 may comprise a light-to-digital sensor or a light-to-frequency sensor. An appropriate light-to-digital sensor for use herein may be a low power ambient light sensor (ALS) or a proximity sensor (PROX). In various embodiments, the sensor for use herein comprises a light-to-digital sensor comprising a photodiode array capable of sensing red, green and blue color light (i.e., an RGB sensor). Of use herein, for example, is a digital red, green and blue color light sensor with an IR blocking filter, such as available from Renesas Electronics Corporation, Tokyo, Japan, under the product number ISL29125. This particular device is packaged at 1.65 mm×1.65 mm square and thus is sized similar to the miniature white LED light source mentioned herein above. In various embodiments, the size of the sensor 130 may be substantially similar to the size of the light source 120. In various embodiments, at least one sensor 130 and at least one light source 120 may be elements in an electronic integrated package. An exemplary package is the TCS3200 color sensor/color detector available from AMS-TAOS—Texas Advanced Optoelectronic Solutions, Inc., Plano, Tex., USA, comprising four LEDs and one optical receiver. These components are generally powered at about 1-6V, and generally from 2.7-5.5V. The power is supplied via the wiring harness 107 discussed herein.

In various embodiments, dressing 100 may comprise a plurality of sensors 130, for example, 2, 3, 4, 5 or more sensors, associated with one or more light sources 120. A plurality of sensors 130 can be positioned to measure relative skin color between the wound site 140 and an unaffected skin surface and/or measure skin color change at the wound site 140 relative to an unaffected skin surface acting as a control. In this regard, data sensed/collected can comprise the skin color change from a baseline along with the difference between a plurality of sensors 130. The rate of the skin color change can also be tracked in accordance with example embodiments of the present disclosure. A plurality of sensors 130 can also provide better resolution to the underlying condition, namely, a skin color gradient of the skin surface. Such a gradient may be particularly instructive in cases of non-linear phlebitis progression.

In various embodiments, such as illustrated in FIG. 1A, a single light source 120 provides the lighting for more than one sensor 130. In other embodiments, a single light source 120 provides the lighting for a single sensor 130. In still other embodiments, more than one light source 120 provide the lighting for a single sensor 130.

With reference now to FIG. 1C, a dressing in accordance with the present disclosure may comprise multiple light sources 120 and multiple sensors 130, such as illustrated in this example wherein there are two of each. In the configuration of FIG. 1C, one set consisting of light source 120a and sensor 130a is positioned so that the wound site 140 is between light source 120a and sensor 130a, while the other set consisting of light source 120b and sensor 130b is distanced from the wound site 140 to provide a reference measure of unaffected skin surface, and optionally a call-out trigger if/when inflammation has reached that far. In various embodiments, the spacing between 120ₐ and 130ₐ (d1) may be equal to the spacing between 120b and 130b (d3), and this spacing may be from about 1-100 mm. In various embodiments, d1 and d3 are chosen independently, considering d1 is at least partly influenced by the dimension of the wound site, whereas d3 may be optimized for baseline skin color readings, irrespective of wound site dimensions. The distance the one light source/light sensor set is spaced apart from the other set varies, and depends on where the surgical or non-surgical site is on the patient (a narrow forearm versus a thigh, for example), the extent to which the progression of the phlebitis is to be detected and monitored, if desired, (wider spacing between the pair would allow for more phlebitis progression before the second set of light source/light sensor detects it), and if there is an unacceptable cross-interference between the sets that needs mitigation. In various embodiments, the second set of light source 120b/light sensor 130b may be used only to obtain a continual baseline skin color reading, without any expectation phlebitis could progress that far, in which case the second set of light source 120b/light sensor 130b may be distanced quite far from the first set of light source 120a/light sensor 130a, even on another limb of the patient and independent of the substrate for the first set of light source 120a/light sensor 130a. In various embodiments, the distance between the two sets of source/sensor may be defined as d4. In various embodiments, the spacing d4 between the two sets of light source/light sensor is about 1-200 mm. In instances when d1 is the same as d3, the four electronic components will appear in a square or rectangular array, depending whether d4=d1 and d3 (square array) or whether d4>d1 and d3 (rectangular array).

In various embodiments, and as illustrated by dashed lines in FIG. 1C, a circuit board 102 may be optionally used to organize the four electronic components, wherein electronic leads 104 may be used to provide power to, and data communication with, the circuit board 102.

In various embodiments, a light source 120 is placed equidistance from (e.g., between) a plurality (e.g., ≥2) of sensors 130, to cut out potential lighting differences. FIGS. 1A and 1B are exemplary of one light source placed equidistance from two light sensors, but the number of light sensors may be >2.

In various embodiments, the dressing 100 can be separately packaged and disposable, wherein the electrical quick-disconnect connector can be separated, the dressing in its entirety discarded, and the electronics (power supply, computer processor, and the portion of the wiring harness) kept for eventual reuse.

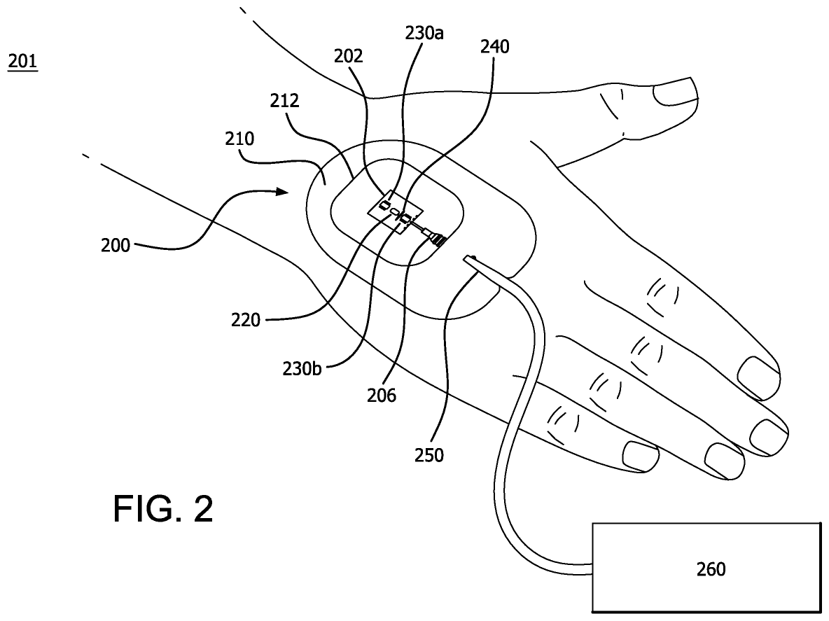

With reference now to FIG. 2, a system for phlebitis detection 201, in accordance with various embodiments, comprises a dressing 200, with its associated components described herein above, and a signal processing device 260 in electronic communication with the dressing 200 via the wiring harness/cable 250. In various embodiments, the signal processor 260 also includes a power supply for the components of the dressing 200. In this way, the cable 250 provides both electrical power to the lights and sensors of the dressing 200 and also facilitates the transfer of data between the dressing 200 and the signal processor 260. In various embodiments, the wiring harness 250 and the signal processing device 260 are not disposable. As mentioned, the disposal portions and the non-disposable portions of the system 201 can be separated by disconnecting the quick-disconnect connector 206.

The wiring harness 250 generally works to transfer measurements from sensor 230a and 230b to the signal processing device 260. In this regard, the wiring harness 250 can comprise a cable or a plurality of wires. In various embodiments, the cable does not include an optical fiber. In various embodiments, the cable does include an optical fiber to transfer light from a light source within device 260 to the dressing 200. The cable 250 can be replaced by a wireless configuration in which data is transferred from the dressing 200 to the processor 260 via short-range radio signals, such as by using Bluetooth technology. In various embodiments, the data transfer across the cable 250 may be digital or frequency responses depending on the nature of the light sensors (i.e., whether light-to-digital or light-to-frequency receivers).

In various embodiments, the signal processing device 260 generally works to receive and process the measurements from the electronic components of the dressing 200, and provide one or more outputs. For example, the signal processing device 260 can compare the measurements to trigger values, and in turn provide one or more outputs.

In various embodiments, an output can be a cue or an automated change to the conditions at a wound site 240. A cue can be a visual indicator (e.g., a red or green light), an audible indicator (e.g., an alarm) or a message sent (e.g., a warning SMS message). In some embodiments, infusion at the wound site 240 can be automatically decreased or stopped. In other embodiments, infusion at the wound site 240 can be automatically increased, for example, so long as the measurements received from the sensors 230a and 230b are acceptable (e.g., below, at, or above a preestablished trigger value). In various embodiments, a treatment at the wound site 240 (e.g., medication, warming or cooling, or compression, etc.) can be automatically delivered to the wound site 240.

In various embodiments of the system illustrated in FIG. 2, an output is provided at one or more light sources 220. For example, a light source 220 can change color or emit a flashing pattern, as controlled by the signal processing device 260. In various embodiments, the light source 220 may comprise an RGB LED rather than a white LED, wherein the signal processing device 260 is used to control the RGB LED output light. In various embodiments, the light source(s) and light sensor(s) of dressing 200 are packaged on a circuit board 202. In these instances, the circuit board may communicate directly with the signal processor 260. In various embodiments, the signal processing device 260 comprises a microcontroller. A circuit board package comprising at least one light source and at least one light sensor may be configured for high-resolution conversion of light intensity to frequency, and configured to communicate digital data with the microcontroller 260 via the cable 250. In non-limiting embodiments, the one or more light sensors 230 may comprise arrays of photodiodes, such as six photodiodes having blue filters, six photodiodes having green filters, six photodiodes having red filters, and six photodiodes having no filters. The photodiodes may be inter-digitated to minimize the effect of nonuniform incident light. In various embodiments, connections on the circuit board 202 may include a GND (ground) pin, a 3-6V supply pin, and three or more data communication or other pins. Other combinations of white LED/color sensor and RBG LED/light sensor may result in different pin configurations for connecting a microcontroller 260 to a circuit board integrating light source(s) and sensor(s) via a cable 250. An exemplary circuit board having two LEDs and one optical sensor, (rather than one LED and two optical sensors), is the Pimoroni 397-PIM412 board comprising two white LEDs on either side of a 6-channel spectral sensor, available from Mouser Electronics, Mansfield, Tex. USA. This example is just to illustrate that LEDs and sensors can be integrated onto small circuit boards, simplifying the connections to the controller 260. Custom circuit boards can be made having the desired number of light sources and light sensors, along with the desired spatial geometries.

In various embodiments, an output is progressive in relation to the progression of the phlebitis being monitored. For example, a light source 220 can exhibit an increased color intensity or flashing frequency, as controlled by the signal processing device 260.

The signal processing device 260 can further comprise a power supply, to supply power to the system (including the light source(s) and sensor(s)). As mentioned, the electrical power supply may be connected via the cable 250 to a circuit board 202, the integrated circuit thereon providing the necessary power to the individual components on the circuit board.

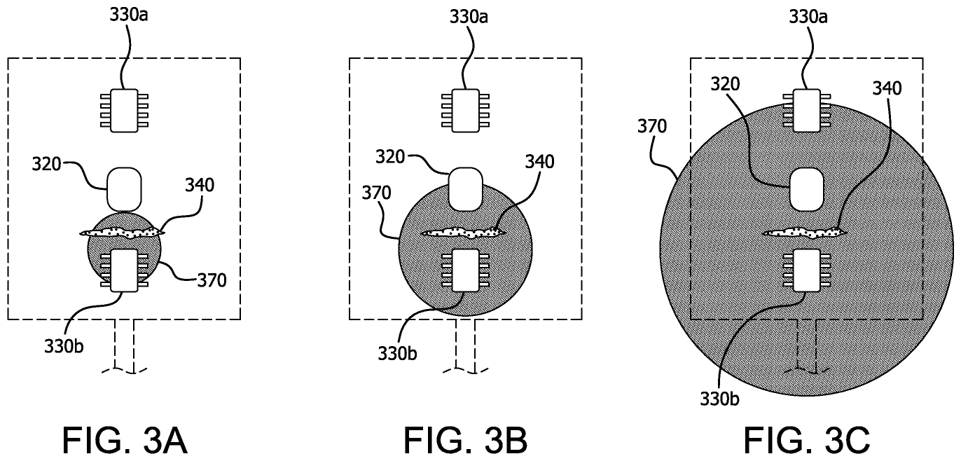

In various embodiments, the present disclosure provides methods for phlebitis detection. In various embodiments, and with reference now to FIG. 3A, a dressing comprising a single light source 320 and two light sensors 330a and 330b evenly spaced apart from the central light course 320, is applied to a wound site 340, such as by adhering the adhesive substrate component of the dressing over the wound 340. As mentioned previously, FIGS. 3A-3C are magnified views of the dressing including only the sensors and the wound site. The dashed lines represent the option that these electronic components are packaged on a circuit board. In FIG. 3A, a light source 320 is illuminated by a power supply, and skin color is measured by a first sensor 330b. In this regard, a baseline skin color can be established based on the patient's skin surface.

As phlebitis 370 progresses (compare the progression from FIG. 3A, to 3B to 3C, indicating a worsening of inflammation), a shift to red skin color, associated with phlebitis 370, is measured by the first sensor 330b and ultimately a second sensor 330a. Data collected can include skin color change from baseline along with the difference between sensors 330b and 330a. The rate of the skin color change can also be tracked. Such measurement, in turn, can be delivered to a signal processing device (e.g., microcontroller 260 in FIG. 2) via a communications cable configured for an appropriate output, as described supra.

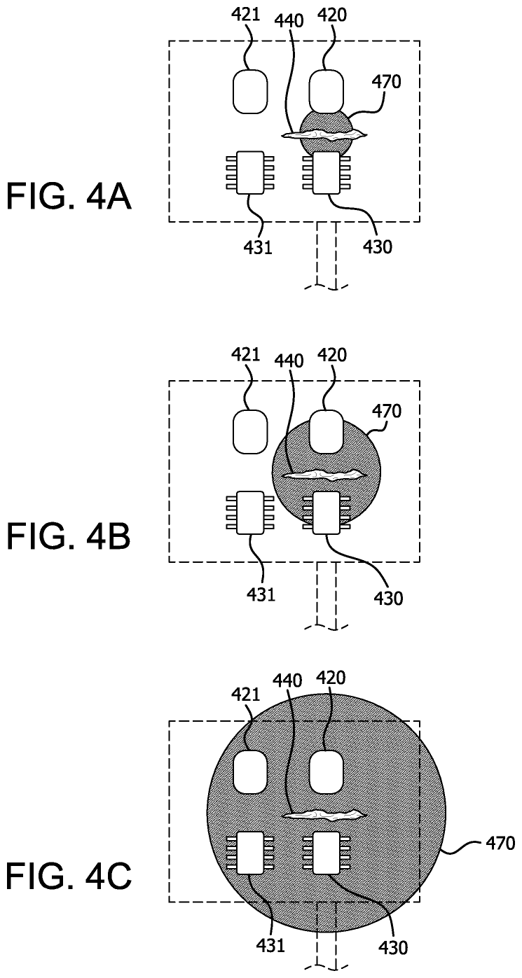

In various embodiments, and with reference now to FIG. 4A, a dressing comprising two light source 420 and 421, and two light sensors 430 and 431, wherein the spacing between 420 and 430 is substantially similar to the spacing between 421 and 431, is applied to a wound site 440, such as by adhering the adhesive substrate component of the dressing over the wound 440. As mentioned previously, FIGS. 4A-4C are magnified views of the dressing including only the sensors and the wound site. The dashed lines represent the option that these electronic components are packaged on a circuit board. A light source 420 is activated, and skin color is measured by a first sensor 430. In this regard, a baseline skin color can be established based on the patient's skin surface.

As phlebitis 470 progresses (compare the progression from FIG. 4A, to 4B to 4C, indicating a worsening of inflammation), a shift to red skin color associated with phlebitis 470 is measured by the first sensor 430 and ultimately a second sensor 431 associated with a second light source 421. As above, data collected can include skin color change from baseline along with the difference between sensors 430 and 431. The rate of the skin color change can also be tracked. Such measurement, in turn, can be delivered to a signal processing device (e.g., microcontroller 260 in FIG. 2) via a communications cable configured for an appropriate output, as described supra.

In the detailed description, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected, coupled or the like may include permanent (e.g., integral), removable, temporary, partial, full, and/or any other possible attachment option. Any of the components may be coupled to each other via friction, snap, sleeves, brackets, clips or other means now known in the art or hereinafter developed. Additionally, any reference to without contact (or similar phrases) may also include reduced contact or minimal contact.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

All structural and functional equivalents to the elements of the above-described various embodiments that are known to those of ordinary skill in the art are expressly incorporated herein by reference and are intended to be encompassed by the present claims. Moreover, it is not necessary for an apparatus or component of an apparatus, or method in using an apparatus to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

1. A dressing comprising:
a substrate configured for placement over a region of skin encompassing a wound site on a patient;
a first light source and a first light sensor fixedly attached to the substrate, the first light sensor spaced apart from the first light source by a distance d1; and
a second light source and a second light sensor fixedly attached to the substrate, the second light sensor separated from the second light source by a distance d3,
wherein the first light sensor is configured to receive light from the first light source reflected off the region of skin in response to the wound site being disposed between the first light source and the first light sensor,
wherein the second light sensor is configured to receive light from the second light source reflected off the region of skin in response to the wound site being disposed between the first light source and the first light sensor and not being disposed between the second light source and the second light sensor, and
wherein the first light source and the first light sensor, and the second light source and the second light sensor, are configured as independent first and second reflectance spectrophotometers, respectively.

2.-18. (canceled)

19. A system for phlebitis detection and monitoring comprising:
a dressing comprising: a substrate configured for placement over a region of skin encompassing a wound site on a patient; a first light source; a first light sensor; a second light source; and a second light sensor, each fixedly attached to the substrate; the first light sensor spaced apart from the first light source by a distance d1; the second light sensor spaced apart from the second light source by a distance d3; and
a signal processing device in electronic data communication with each of the first light source, the first light sensor, the second light source and the second light sensor,
wherein the first light sensor is configured to receive light from the first light source reflected off the region of skin in response to the wound site being disposed between the first light source and the first light sensor, and the second light sensor is configured to receive light from the second light source reflected off the region of skin in response to the wound site being disposed between the first light source and the first light sensor and not being disposed between the second light source and the second light sensor,
wherein the second light sensor is configured to measure a baseline skin color of the region of skin, and wherein the first light sensor is configured to measure a change in skin color relative to the baseline skin color,
wherein the first light source and the first light sensor, and the second light source and the second light sensor, are configured as independent first and second reflectance spectrophotometers, respectively,
wherein the signal processing device provides an output in response to a change from the baseline skin color, and
wherein the output comprises at least one of a visual indicator, an audible indicator, a message sent, and an automated change to the conditions at the wound site.

20. (canceled)

21. A method of detecting and monitoring the rate of phlebitis at a region of skin encompassing a wound site on a patient, the method comprising:
covering the region of skin with a dressing comprising a first light source and a first light sensor spaced apart from the first light source by a distance d1 and a second light source and a second light sensor spaced apart from the second light source by a distance d3, such that the wound site is disposed between the first light source and the first light sensor and not disposed between the second light source and the second light sensor; and
obtaining a color of a portion of skin in the region of skin between the first light source and the first light sensor by measuring light from the first light source reflected off the portion of skin over time relative to a baseline skin color,
wherein the dressing further comprises a substrate dimensionally configured to cover the region of skin and onto which the first light source, and the first light sensor, the second light source and the second light sensor are fixedly attached; wherein the first light source and the first light sensor, and the second light source and the second light sensor, are configured as independent first and second reflectance spectrophotometers, respectively,
wherein a red color thus obtained by the first reflectance spectrophotometer indicates the presence of phlebitis, and
wherein a reddening of skin color over time relative to the baseline skin color indicates a worsening of the phlebitis.

22.-35. (canceled)

36. The dressing of claim 1, wherein at least one of:
the first reflectance spectrophotometer is configured to measure a color of the region of skin;
the first light source comprises a white LED or a full-color RGB LED lamp;
the first light sensor comprises an RGB color sensor;
the substrate comprises an adhesive portion configured to adhere the dressing to the patient;
the substrate comprises a transparent region dimensionally sized to incorporate the first light source, the first light sensor, and the region of skin within its dimensions;
d1 is from about 1 mm to about 100 mm;
the first light source and the first light sensor are integrated on a circuit board having a front face whereupon the first light source and the first light sensor are exposed and a back face that is fixedly attached to the substrate;
the second reflectance spectrophotometer is configured to measure a baseline skin color of the region of skin, and wherein the first reflectance spectrophotometer is configured to measure a change in skin color relative to the baseline skin color;
the second light source comprises a white LED or a full-color RGB LED lamp;
the second light sensor at least one of comprises an RGB color sensor, or is configured to measure a baseline skin color of the region of skin, and wherein the first light sensor is configured to measure a change in skin color relative to the baseline skin color; or
the second light source and the second light sensor are integrated on a circuit board having a front face whereupon the first light source and the first light sensor are exposed and a back face that is fixedly attached to the substrate.

37. The dressing of claim 1, further comprising a wiring harness configured to supply power to, and data communication with, the first light source and the first light sensor, the wiring harness further including a quick-disconnect connector along the wiring harness, adjacent the substrate.

38. The dressing of claim 37, wherein the wiring harness is further configured to supply power to, and data communication with, the second light source and the second light sensor.

39. The dressing of claim 1, further comprising an additional light sensor fixedly attached to the substrate and spaced apart from the first light source by a distance d2, the additional light sensor positioned on the opposite side of the first light source from the first light sensor such that the additional light sensor, the first light source, and the first light sensor are linearly aligned, wherein the additional light sensor is configured to receive light from the first light source reflected off the region of skin in response to the wound site being disposed between the first light source and the first light sensor and not being disposed between the first light source and the additional light sensor.

40. The dressing of claim 39, wherein at least one of:
the additional light sensor is configured to measure a baseline skin color of the region of skin, and wherein the first light sensor is configured to measure a change in skin color relative to the baseline skin color;
the distance d2 is equal in length to the distance d1; or
each distance d1 and d2 is independently from about 1 mm to about 100 mm.

41. The dressing of claim 1, wherein the distance d3 is equal in length to the distance d1, and wherein the second light source and the first light source are separated by a distance d4, and the second light sensor and the first light sensor are separated by the same distance d4, such that the first and second light sources and the first and second light sensors are disposed in a square or a rectangular array.

42. The dressing of claim 41, wherein the distance d4 is from about 1-200 mm.

43. The system of claim 19, wherein the first light source, the first light sensor, and the second light source, and the second light sensor are integrated on a circuit board, the circuit board electrically powered by a power supply disposed in the signal processing device.

44. The method of claim 21, wherein at least one of:
the first light source comprises a white LED or a full-color RGB LED lamp; or
the dressing further comprises an additional light sensor fixedly attached to the substrate and spaced apart from the first light source by a distance d2, the additional light sensor positioned on the opposite side of the first light source from the first light sensor such that the additional light sensor, the first light source, and the first light sensor are linearly aligned, wherein the additional light sensor is configured to receive light from the first light source reflected off the region of skin in response to the wound site being disposed between the first light source and the first light sensor and not being disposed between the first light source and the additional light sensor.

45. The method of claim 44, wherein the distance d1 is equal to the distance d2.

46. The method of claim 21, further comprising at least one of:
measuring the baseline skin color of a portion of skin in the region of skin between the first light source and the additional light sensor, and measuring a color of the portion of skin in the region of skin between the first light source and the first light sensor over time relative to the baseline skin color; or
measuring a baseline skin color of a portion of skin in the region of skin between the second light source and the second light sensor, and measuring a color of the portion of skin in the region of skin between the first light source and the first light sensor over time relative to the baseline skin color.

47. The method of claim 21, wherein the distance d3 is equal in length to the distance d1, and wherein the second light source and the first light source are separated by a distance d4, and the second light sensor and the first light sensor are separated by the same distance d4, such that the first and second light sources and the first and second light sensors are disposed in a square or a rectangular array.
